(19) United States
(12) Patent Application Publication
(10) Pub. No.: US 2006/0058874 A1
(43) Pub. Date: Mar. 16, 2006

(54) Double bifocal intraocular lens-spectacle telescopic device for low vision use

(76) Inventor: Eliezer Peli, Newton, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/526,740

(22) PCT Filed: Sep. 4, 2003
(86) PCT No.: PCT/US03/27642

§ 371 Date: Mar. 4, 2005

Related U.S. Application Data
(60) Provisional application No. 60/408,191, filed on Sep. 4, 2002.

(51) Int. Cl. A61F 2/16; G02C 7/04; G02C 1/00; G02C 7/02

(52) U.S. Cl. 623/6.28; 623/6.35; 351/160 R; 351/158; 351/159

(57) ABSTRACT

A double bifocal intra-ocular lens and spectacle system that can be used to enhance the vision of persons suffering from low-vision includes a first optical element placed in or on the eye, and a second optical element placed external to the eye. In particular, the first optical element is designed to be placed in or on the eye. This element includes an inner region and an outer annular region with first and second optical powers respectively. A second optical element that has two regions is disposed external to the eye and aligned so that a first region having a third optical power is optically coupled to the outer annular region for normal vision and the respective optical powers are selected to provide for the standard distance correction of the eye. The second region is arranged so that when aligned with the eye, the second region and the inner region of the first optical element form a Galilean telescope that provides for a magnified image to be provided therefrom.

### CROSS REFERENCE TO RELATED APPLICATIONS

This application claims the benefit of U.S. Patent Application No. 60/408,191, filed on Sep. 4, 2002, the whole of which is hereby incorporated by reference.

### STATEMENT REGARDING FEDERALLY SPONSORED RESEARCH OR DEVELOPMENT

N/A

### BACKGROUND OF THE INVENTION

This invention relates generally to vision correction systems and in particular to vision correction systems for patients having impaired vision, e.g., low vision, caused by age or disease.

One of the leading causes of blindness in adults is a disease of the eye known as macular degeneration. Macular degeneration generally affects the central portion of the retina known as the macula, which is an area of the eye that processes images focused by the cornea and the lens. This portion of the eye provides a person's acute vision. Although only a small portion of the retina is affected, typically between 1-5%, this degeneration of the macula can lead to vision loss to the level of 20/200 or worse. Thus, driving and reading can be adversely affected while peripheral vision remains intact. This condition is commonly referred to as low vision. There are a number of other diseases that cause visual loss of similar nature.

There is currently no specified treatment to reverse the effects of macular degeneration, and in the absence of effective treatments, other optical and electronic based systems are used to provide assistance in overcoming the low vision effect. For example, telescopic systems that attach to a patient's spectacles increase the retinal image size of a given distant object when viewed through the spectacles. However, telescopic systems reduce a patient's visual field to approximately 11 to 14° (for a 3× magnification), which greatly restricts the patient's range of activities of a patient.

The problems associated with a reduced visual field have been partially overcome by implanting a portion of the telescopic system within the eye of the patient. These systems may provide up to 3× magnification but with a wider field of view than telescopic systems that are entirely external to the eye. However, as pointed out in a publication by Baily, 1987, “Critical view of an ocular telephoto system,” Contact Lens Association of Ophthalmologists Journal, 13(4):217-221, the instantaneous field-of-view is only slightly wider than that achieved with a spectacle mounted telescope. Further, the field-of-fixation of these systems is also limited because these systems stabilize the retinal image such that eye movements will result in minimal image movement on the retina (Doesschate J T, De Vries H. A method of obtaining the image of a light source on a fixed spot of the retina, independent of fixation movements. Ophthalmologica 1948;127:65-73. Drasdo N. The effect of high-powered contact lenses on the visual fixation reflex. Br J Physiol Opt 1970;25:14-22.). An improvement of this basic idea includes the bifocal IOL design implemented by Allergan in which the IOL central portion contains the high minus lens and the periphery contains the normal IOL pseudo-phakic correction. This design permits using the IOL either with the high power spectacle lens as a telescope or with normal pseudo-phakic correction without the magnification and with a wider field of view than in the telescopic configuration. A clinical trial conducted by Allergan proved that the latter use is possible, and no problems were reported with the IOL as a result of the highly out-of-focus image formed by the negative segment of the lens. (see, e.g., Koziol, J. E., Peyman, G. A., Cionni, R., et al. (1994). “Evaluation and implementation of a teledioptric lens system for cataract and age-related macular degeneration,” Ophthalmic Surgery 25: 675-684.).

However, in all reported studies (see, e.g., Koziol, J. E., Peyman, G. A., Cionni, R., et al. (1994). “Evaluation and implementation of a teledioptric lens system for cataract and age-related macular degeneration,” Ophthalmic Surgery 25: 675-684; and Garnier, B., and Colonna De Lega, X. (1992). “Low-vision aid using a high minus intraocular lens,” Applied Optics 31:3632-3636), patients either did not benefit from the telescope in the spectacle lens portion of the system, or they refused to use the high power spectacle component at all. This might be a result of the unacceptable cosmetics of the large high power lens or a result of the limited field-of-fixation, or both.

One solution has been to use a combination of bifocal contact lenses and spectacles. A combined contact lens/spectacle telescope was described in 1936 by Dallos (Dallos J. Contact glasses, the invisible eye glasses. Archives of Ophthalmology 1936;15:617-23), and it was introduced as a low vision device soon thereafter (see, Ludlam W M. Clinical experience with the contact lens telescope. Am J Optom 1960;37:363-72, for a review of early results). There are also two varieties of the combined contact lens/spectacle design, one with a single power contact lens and the other with a bifocal contact lens, (see Filderman I P. The telecon lens for the partially-sighted. Am J Optom and Arch Am Acad of Optom 1959;36:135-6). In the former design, a high negative power contact lens in combination with a high positive power spectacle lens provides magnification. Moore realized the visual field limitation of the device and suggested that this device would be useful only for a patient with a minimal need for peripheral vision, but he also believed that the best use would be monocular with the other eye used for peripheral vision (binocular multiplexing). Moore indicated also that this design did not solve the cosmesis problem of the spectacle telescope as patients often rejected the device because of the thick unsightly large high power spectacle lens, (see Moore L. The contact lens for subnormal visual acuity. Optics 1964;21:203-4). In the bifocal design, the contact lens is a concentric bifocal with the outer segment providing a standard contact lens power and the central zone of the anterior surface is flat providing the high negative power (see, Filderman I P. The telecon lens for the partially-sighted. Am J Optom and Arch Am Acad of Optom 1959;36:135-6). Filderman developed a bifocal spectacle lens to combine with the bifocal contact lens. In the bifocal spectacle lens of Filderman, the smaller concentric high power lens was centrally mounted in the spectacle lens and was aligned with the pupil in the primary position of gaze. The carrier piano lens was to be used together with the outer segment of the contact lens for peripheral vision with no magnification while the smaller concentric high power inset lens, when combined with the negative power segment of the contact lens, was designed to provide the magnification with a reduced central field. Filderman recommended monocular use of his system to permit biocular multiplexing and he felt that the cosmetic advantage of this design was substantial to justify its use in many cases, (See Filderman I P. The telecon lens for the partially-sighted. Am J Optom and Arch Am Acad of Optom 1959;36:135-6 and Filderman I P. The telecon lens system, a modified Galilean telescope. Contacto 1959;3:94-6).

In general, the only telescopic aids successfully used for low vision today are those used intermittently. These include hand-held telescopes and bioptic telescopes mounted above the line of sight and used only about 5-10% of the time, even in the most intense situation of driving. Although sometimes spectacle-mounted telescopes are centrally mounted in the carrier lens, they are typically used for fixed task such as computer use, playing music, etc. The reason for this is that the patient using a telescope wishes to benefit from the magnification option when this is needed for fine details and from the wide field of the unmagnified view when needed for safe navigation. This latter requirement has not been met by the IOL (or contact lens) telescopic aids implemented to date. Even if the patient has a second functioning eye, it may be impossible for that person to use two images so widely different in magnification.

Therefore, it would be advantageous to provide an IOL telescopic aid that overcomes the problems of existing IOL telescopic aids described above.

### BRIEF SUMMARY OF THE INVENTION

A double bifocal intraocular-lens (IOL) spectacle system for providing enhanced vision for people having low vision is disclosed. In one embodiment, a first optical element is configured to include an outer annular region with a first optical power and an inner portion having a second optical power. The first optical element is configured to be implanted within the eye as an IOL or placed on the eye as a contact lens. A second optical element is disposed external to the eye and includes a lower region and an upper region. The second optical element is oriented so that the lower region is aligned with the pupil of the eye for normal viewing. The lower region has a portion having a third optical power that is selected with the first optical power of the outer annular region to provide for the standard distance correction required by the eye. The upper region has a portion having a fourth optical power that is selected with the second optical power of the inner region to provide a magnified retinal image. This allows a user to shift their gaze from the lower region to the upper region in order to increase the magnification of an object to help the user examine previously unresolvable detail. In general, the fourth optical power is a positive optical power and the second optical power of the inner region is a negative optical power such that the combination of the two optical elements forms a Galilean telescope.

Another embodiment of the present invention includes the same first optical element as the first embodiment, including inner and outer annular regions having first and second optical powers, respectively. In this embodiment, the second optical element includes middle, upper, and lower regions. The second optical element is configured such that the middle region is aligned with the pupil of the eye under normal viewing. The middle region has a portion having a third optical power that is selected with the first optical power of the outer annular region to provide for the standard distance correction required by the eye. The upper region has a portion having a fourth optical power that is selected with the second optical power of the inner region to form a first Galilean telescope. The first Galilean telescope is configured to provide an afocal magnified retinal image. The lower region has a portion having a fifth optical power that is selected with the second optical power of the inner region to form a second Galilean telescope. The second Galilean telescope is configured to provide a focal length that is closer to the patient so that it forms a larger retinal image than that provided by first Galilean telescope formed in the upper region. In this way, the second Galilean telescope has a larger apparent magnification than the first Galilean telescope. The upper region thus provides an image for viewing distant objects while the lower region provides an image for viewing closer objects such as books for reading.

Other features, functions, and aspects of the invention will be evident from the Detailed Description of the Invention that follows.

### BRIEF DESCRIPTION OF THE SEVERAL VIEWS OF THE DRAWING

The invention will be more fully understood with reference to the following Detailed Description of the Invention in conjunction with the drawings of which:

FIG. 1A is a side sectional view of the present invention in a first position allowing a user to view unmagnified wide field images;

FIG. 1B is a side sectional view of the present invention in a second position allowing a user to view magnified narrow field images;

FIG. 2A is a side sectional view of another embodiment of the present invention in a first position allowing a user to view unmagnified wide field images; and

FIG. 2B is a side sectional view of the present invention in a third position allowing a user to view a more magnified field images than in a corresponding second position;

### DETAILED DESCRIPTION OF THE INVENTION

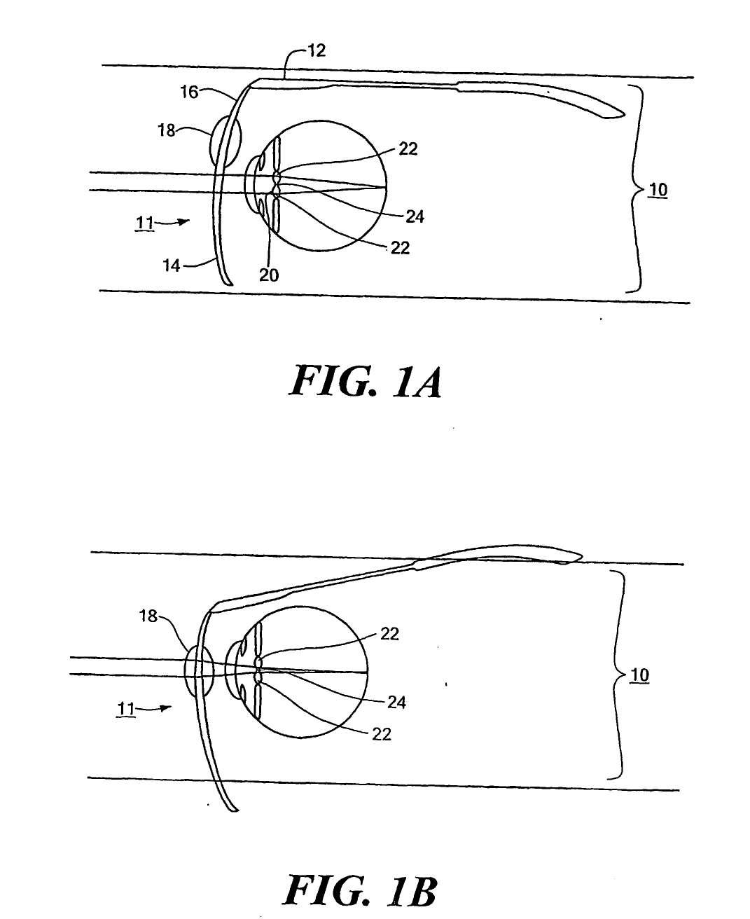

In a preferred embodiment of the bifocal telescopic intraocular-lens (IOL) spectacle device according to the invention, an intraocular lens having a first optical configuration operates in conjunction with a spectacle lens having a second optical configuration to provide for two or more modes of vision that are suitable for normal and enhanced vision for people having low vision disorders. As depicted in FIG. 1A, a telescopic bifocal IOL-spectacle device 10 includes an IOL 20 and a spectacle carrier lens 11 in optical communication with one another.

In particular, the IOL 20 includes an outer annular region 22 having a first optical power and an inner region 24 having a second optical power. FIGS. 1A and 1B further depict the spectacle carrier lens 11 with lower and upper regions 14 and 16 respectively. In particular, the lower region 14 includes the center portion of the carrier lens that is aligned with the pupil of the eye in the primary position of gaze. The lower region 14 can include a portion having a third optical power or may be a blank. The upper region 16 is an upper region of the spectacle carrier lens 11. In particular, the upper region 16 includes the upper portion of the carrier lens and is not aligned with the pupil of the eye in the primary position of gaze. The upper region 16 further includes a lens 18 having a fourth optical power. As will be explained in more detail below, the lens 18 is typically a positive power lens that is used in forming the IOL-telescopic system in conjunction with the inner region of the IOL 20.

In a first vision mode, the optical portion of the lower region 14 of the carrier lens 11 is configured and arranged to be: in optical communication with the annular region 22 of the IOL 20. In this mode that is used for normal (unmagnified) viewing by the patient, the optical combination of the optical portion of the first region 14 and the outer region 22 of the IOL 20 provide for normal wide-angle unmagnified viewing of desired objects. Accordingly, the third and first optical powers of the optical portion within the lower region 14 of the carrier lens 11 and the annular region 22, respectively, are selected so that the combination of the two optical powers provides for standard distance pseudophakic correction.

In the event that a patient using the present invention observes an object with unresolvable details, the invention provides a second vision mode. The patient is able to tilt his head and thus to shift his gaze so that the lens 18 within the upper segment 16 is aligned with the pupil and is optically coupled to the inner region 24 of the IOL 20. In general, the inner region 24 of the IOL 20 is configured as a lens having a negative optical power and the lens 18 within the upper segment 18 of the spectacle 11 has a positive optical power. The lens 18 within the upper region 16 and the inner region lens 24 of the IOL 20 are configured and arranged to provide clear vision through the cornea and lens 24, and thus form a Galilean telescopic system. The Galilean telescope provides a higher power magnification view of an object, but with a concomitant reduction in the field of view. Typically, the power of the telescope can be varied up to 3.0×. The inner lens 24 can range in power from −30 diopters to −100 diopters. The power of the lens 18 in the upper region 16 of the carrier lens 11 has to be adjusted individually for a patient based on their refractive error and would vary based on the power of the negative lens. For a typical patient the power of lens 18 will vary from +10 to +30 diopters.

Advantageously, the first vision mode provides for a wider field of view for comfortable safe vision required for mobility and navigation, while the second vision mode provides for increased magnification required for closer inspection of the details of the observed object. Typically, the patient alternates between the first and second vision modes by redirecting his gaze through the lens 18 with a tilt of his head through a small angle. This configuration advantageously also provides cosmetically acceptable correction that is only slightly different in appearance from normal bifocal lenses. An important cosmetic consideration for the wearer is that the person's eyes for the most part are seen through a normal spectacle lens when he is conversing with other people.

The placement of the lens 18, however, limits the vertical size of the lens. The lower limit of the lens 18 is provided by the pupil and the upper limit of the lens 18 is provided by the spectacle lens. Thus, to maximize the amount of light collected by the lens, the lens 18 should be as elongated in the horizontal dimension as far as possible. This maximizes the brightness of the magnified image.

Another aspect of the present invention is that the patient's prescription can be incorporated onto both the lower region 14 and the upper region 16 of the spectacle carrier lens 11. Thus, a person requiring astigmatic or spherical correction separate from the spherical correction required by the telescopic system can be accommodated. This allows individuals to use the present invention without losing the benefit of the increased magnification due to the increased blur when the upper region 16 is uncorrected for their particular prescription.

In another aspect of the invention, the lens 18 should be designed to have a simple appearance, e.g., the lens 18 should be a single high power lens. Although multiple lens systems can be used in the present invention and may provide for a wider field of view, in general, multiple lens systems are not acceptable cosmetically and therefore are not utilized as often by patients. In addition, the overall benefits of a multiple lens system may not be as useful in the present invention, because of the limitation on the field of fixation. In some instances, a lower quality lens may be acceptable to an individual in consideration of his poor visual acuity. Also, because eye movements that permit scanning are not useful with the present invention, the quality of the image across a wide field of view is not as important.

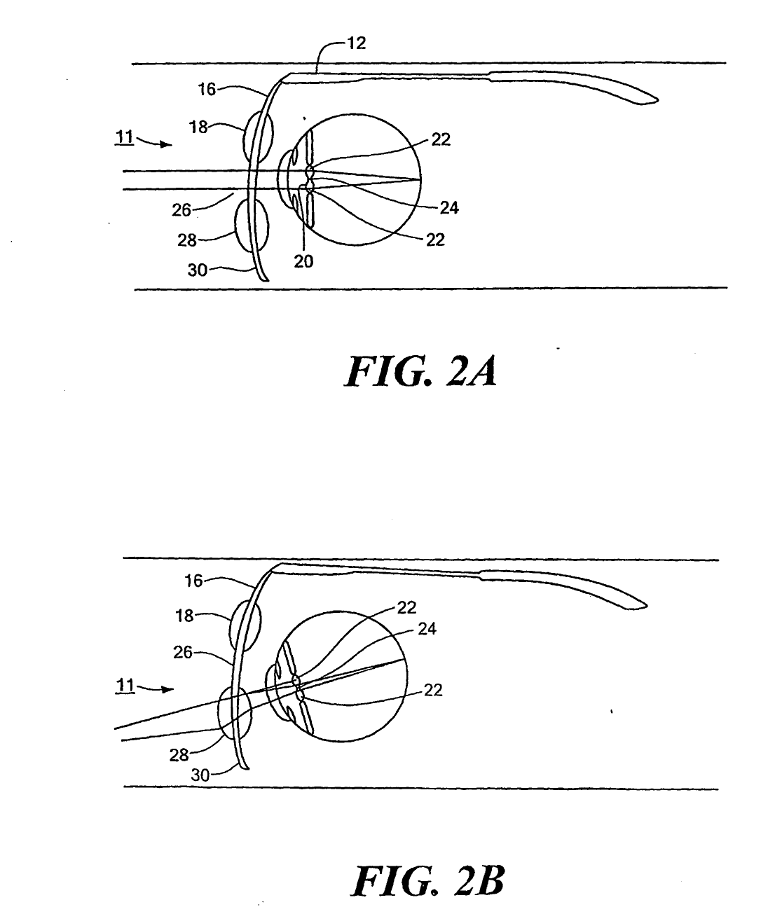

Another embodiment of the present invention that provides for more than two vision modes is depicted in FIGS. 2A and 2B. In this embodiment, the IOL 20 is as described above, having an outer region 22 and an inner region 24, each having first and second optical powers respectively. In this embodiment, the spectacle carrier lens 11 includes a lower region 30, an upper region 16, and a middle region 26. The middle region 26 includes an optical portion having a third optical power, the upper region 16 includes a first lens 18 having a fourth optical power and the lower region 30 includes a second lens 28 having a fifth optical power.

As discussed above, in a first vision mode, the optical portion of the middle region 26 is configured and arranged to be in optical communication with the annular region 22 of the IOL 20 for normal viewing by the patient. The optical combination of the optical portion of the middle region 26 and the outer region 22 of the IOL 20 is used for normal, wide-angle, unmagnified viewing of desired objects. Accordingly, the third and first optical powers of the optical portion within the lower region 30 and the annular region 22, respectively, are selected so that the optical combination of the two optical powers provides for standard distance pseudophakic correction.

Also as discussed above, a second vision mode for a patient is provided using the present invention. The patient is able to shift his gaze so that the lens 18 within the upper segment 16 is optically coupled to the inner region 24 of the IOL 20. In general, the inner region 24 of the IOL 20 is configured as a lens having a negative optical power and the lens 18 within the upper segment 18 has a positive optical power. The lens 18 within the upper region 16 and the inner region lens 24 of the IOL 20 are configured and arranged to provide clear vision through the cornea and the lens 24 and thus to form a first Galilean telescope. The first Galilean telescope provides a higher power magnification of an object, but with a concomitant reduction in the field of view. The first Galilean telescopic system formed using the inner region 24 of lens 20 in conjunction with the lens 18 in the upper region 16 of the carrier lens 11 provides a high power magnification of a distant object than the standard distance pseudophakic correction of the middle section 26 and the outer section 22 of lens 20, and with the concomitant reduction in the field of view and the restriction of the field of fixation. In general, the first Galilean telescope system is an afocal system, i.e., a system adjusted to have object and image points at infinity. Accordingly, it is usually not necessary to adjust the optical power of the lens 16 once the first Galilean telescopic system is set.

However, in some circumstances, particularly in reading where magnification may be required to permit a relatively large reading distance, this embodiment of the present invention provides a third vision mode providing for a second level of increased magnification. Lens 28 in the lower region 30 of the carrier spectacle lens 11 is configured and arranged in conjunction with the inner region 24 of the IOL 20 to form a second Galilean telescope which is focused at a comfortable reading distance (ie 25 cm). The lens 28, however, is provided with an increase in power over the lens 18, typically on the order 3-6 diopters, to provide for a shorter focal distance than the first (afocal) Galilean system. In general, the magnification of the second Galilean system can be adjusted by a clinician for an individual patient. In particular, the magnification can be adjusted by adding or subtracting power to/from lens 28 to alter the focal distance of the second Galilean telescope to take into account the particular needs of a patient. Thus, as the vision of a patient gets worse or otherwise changes, the focal length of the second Galilean telescopic system can be adjusted accordingly.

As discussed above with respect to the upper lens 18 in the first embodiment, the lens 18 in this embodiment is also limited in size in the vertical dimension since the lens 28 is not to be in the primary gaze of the patient. Thus, the lens 18 can extend no lower than the bottom of the pupil and no higher than the spectacle frame. Accordingly, in one embodiment, the lens 18 is elongated in the horizontal dimension to maximize the amount of light collected by the lens and thereby maximize the brightness of the magnified image.

Similarly, the lower lens 28 is limited in size in the vertical dimension since it is not to be in the primary gaze of the patient. Thus, the second lens can extend no higher than the bottom of the pupil and no lower than the spectacle frame. Accordingly, in one embodiment, the lens 28 is elongated in the horizontal dimension to maximize the amount of light collected by the lens and thereby maximize the brightness of the magnified image.

The other aspects of the invention discussed with respect to the first embodiment are also to be considered aspects of this embodiment as well, and those descriptions are not repeated.

Although the embodiments described above include a description of a single spectacle carrier lens, the present invention provides additional advantages when used in a binocular configuration. During the use of the standard pseudophakic segment, i.e., the spectacle regions that are not occupied by the lens 18 or 28 that include the lower region 14 of the carrier lens 11 of the first embodiment and the middle region 26 of the second embodiment, allow binocular vision to be used naturally by the patient. This allows the patient to achieve the higher acuity and wider field of view, e.g., centrally with partially non-overlapping scotomas and peripherally at all times. When used with the telescopic portion of the carrier lens placed on both sides of the spectacle frame, binocular vision may or may not be possible. The main difficulty is in achieving the proper alignment, both vertically and horizontally, of the two sides of the spectacle. In the event that proper alignment cannot be achieved, or is not able to be stabilized, one of the telescopic sections can be removed for one eye. The telescopic section may also be switched between eyes as the eyesight of the patient changes due to disease progress or age.

The lenses 18 and 28 described above can be formed as integral parts of the spectacle carrier lens 11 or may be specially designed stick-on lenses. In addition, the power of the lenses 18 and 28 may be adjusted by either using additional specially designed stick-on lenses or by removing and replacing the original stick-on lens with a replacement stick-on lens. The lenses 18 and 28 can also be inserted into the carrier lenses, rather than sticking them on to it.

In another variation, the IOL 20 in the above described embodiments can be replaced with a bifocal contact lens having a configuration similar to the IOL 20. That is, the bifocal contact lens should include an inner portion that has the high negative power correction and an outer annular region providing the standard distance pseudophakic correction.

In another variation, the carrier lens 11 can be a lens blanc in which the individual prescription (spherical and astigmatic) is placed on the back surface of the lens, substantially covering the back surface. In this variation, the lenses 18 and 28 will not affect the prescription.

Placing the prescription of the patient on the back surface of the carrier lens 11 provides advantages in manufacturing these lenses. A lens blanc can be molded with upper, or upper and lower, regions having the requisite optical power as described above. The prescription is then formed on the back of the carrier lens and provides for corrected telescopic viewing as well as corrected normal viewing.

Those of ordinary skill in the art should further appreciate that variations to and modification of the above-described apparatus and system for providing a bifocal telescopic IOL-spectacle device may be made without departing from the inventive concepts disclosed herein. For example, the foregoing embodiments may be disposed in a kit complete with instructions on the application and use of the parts included therein. Accordingly, the invention should be viewed as limited solely by the scope and spirit of the appended claims.

What is claimed is:

1. An apparatus for improving low vision in the eye, comprising:
a first optical element including an outer annular region having a first optical power and an inner region having a second optical power, said optical element sized and configured for placement on or within the eye;
a second optical element external to the eye and sized and configured to be in optical communication with said first optical element, said second optical element including first and second regions, said first region having a first portion having an optical power selected in conjunction with said first optical power of said outer region to provide for substantially the standard distance correction required by the eye, said second region including a second portion having an optical power selected in conjunction with said inner region of said first optical element to provide a magnified focused retinal image.

2. The apparatus of claim 1 wherein said second optical element is a spectacle lens contained within a spectacle frame.

3. The apparatus of claim 1 wherein said inner region of said first optical element is a first lens having a negative optical power and said second portion of said second region includes a second lens having a positive optical power sufficient to compensate for said negative power and creating a clear (in focus) retinal image, thus forming a Galilean telescopic system.

4. The apparatus of claim 3 wherein said inner region of said first optical element has a negative optical power between −30 and −100 diopters.

5. The apparatus of claim 3 wherein said second region of said second optical element has a positive optical power between +10 and +40 diopters.

6. The apparatus of claim 1 wherein said outer annular region of said first optical element is substantially equal to the required pseudophakic correction of the corresponding eye within which said first optical element is implanted and said second region has an optical power substantially equal to zero.

7. The apparatus of claim 1 wherein said outer annular region of said first optical element is in optical communication with said second region of said second optical element and wherein said optical power of said outer annular region and said optical power of said second region are selected to be substantially equal to the required correction of the corresponding eye within which said first optical element is implanted.

8. The apparatus of claim 2 wherein said first region of said spectacle lens is a lower region of said carrier lens, and includes substantially a central region of said spectacle carrier lens and said second region of said spectacle lens is an upper region of said spectacle lens.

9. The apparatus of claim 8 wherein said second portion of said upper region includes a lens having a horizontally elongated configuration, wherein said horizontally elongated configuration is sized and configured to provide as wide a field of view as practical.

10. The apparatus of claim 2 wherein the second portion of said second region is a lens.

11. The apparatus of claim 10 wherein said lens is a single element lens.

12. The apparatus of claim 11 wherein said lens is formed integral with said spectacle carrier lens.

13. The apparatus of claim 11 wherein said lens is a stick-on lens applied to the surface of said spectacle carrier lens.

14. The apparatus of claim 11 wherein said lens is inserted into said spectacle carrier lens.

15. The apparatus of claim 2 wherein said carrier lens includes an optical prescription to correct the vision of the corresponding eye.

16. The apparatus of claim 15 wherein the optical prescription to correct the vision of the corresponding eye is placed on a back surface of said carrier lens.

17. The apparatus of claim 15 wherein the optical prescription to correct the vision of the corresponding eye is placed on a front surface of said carrier lens.

18. The apparatus of claim 1 wherein the first optical element is an intraocular lens for implantation within the eye.

19. The apparatus of claim 1 wherein the first optical element is a contact lens for placement on the surface of the eye.

20. The apparatus of claim 1 wherein the first optical element is a pair of optical elements, one for each eye, and wherein the second optical element is a pair of second optical elements one for each eye, wherein binocular vision is provided for in both the unmagnified wide-angle view and the magnified retinal image view.

21. The apparatus of claim 1 wherein the first optical element is a pair of optical elements, one for each eye, and wherein the second optical element is a single second optical element, wherein binocular vision is provided for in the unmagnified wide-angle view and monocular vision is provided in the magnified retinal image view.

22. An apparatus for improving low vision in the eye, comprising:
a first optical element including an outer annular region having a first optical power and an inner region having a second optical power, said optical element sized and configured for placement on or within the eye;
a second optical element external to the eye and sized and configured to be in optical communication with said first optical element, said second optical element including first, second, and third regions, said first region having a portion having an optical power selected in conjunction with said outer region of said first optical element to provide for substantially the standard distance pseudophakic correction of the eye, said second region having a portion having an optical power selected in conjunction with said inner region of said first optical element to provide a focused retinal image having a first magnification of a distant object, and said third region having a portion having an optical power selected in conjunction with said inner region of said first optical element to provide a clear retinal image having a second magnification of an object at a short (reading) distance.

23. The apparatus of claim 22 wherein said second optical element is a spectacle lens contained within a spectacle frame.

24. The apparatus of claim 22 wherein said inner region of said first optical element is a first lens having a negative optical power and said second region of said second optical element includes a second lens having a positive optical power, each of said first and second lenses having a focal point, said first and second lenses being configured and arranged to create a clear (in focus) retinal image, thus forming a Galilean telescopic system., having said first magnification.

25. The apparatus of claim 22 wherein said inner region of said first optical element is a first lens having a negative optical power and said third region of said second optical element includes a third lens having a positive optical power, each of said first and third lenses having a focal point, said first and third lenses being configured and arranged to sufficient to compensate for said negative power and creating a clear (in focus) retinal image, thus forming an intraocular Galilean telescope having said second magnification.

26. The apparatus of claim 24 or 25 wherein said inner region of said first optical element has a negative optical power between −30 and —100 diopters.

27. The apparatus of claim 24 or 25 wherein said second region of said second optical element has a positive optical power between +12 and +55 diopters.

28. The apparatus of claim 25 wherein said third region of said second optical element has a positive optical power between 2 and 15 diopters greater than said second region of said second optical element.

29. The apparatus of claim 22 wherein said outer annular region of said first optical element is substantially equal to the optical power required for pseudophakic correction of the corresponding eye within which said first optical element is implanted.

30. The apparatus of claim 22 wherein said outer annular region of said first optical element is in optical communication with said second region of said second optical element and wherein said optical power of said outer annular region and said optical power of said second region are selected to be substantially equal to the required correction of the corresponding eye within which said first optical element is implanted.

31. The apparatus of claim 23 wherein said first region of said first region is a middle region of said spectacle lens, said second region of said spectacle lens is an upper region of said spectacle lens, and said third region is a lower region of said spectacle lens.

32. The apparatus of claim 31 wherein said second portion of said upper region of said spectacle lens includes a lens having a horizontally elongated configuration, wherein said horizontally elongated configuration is sized and dimensioned to provide as wide a field of view as is practical.

33. The apparatus of claim 31 wherein said third portion of said lower region of said spectacle lens includes a lens having a horizontally elongated configuration, wherein said horizontally elongated configuration is sized and dimensioned to provide as wide a field of view as is practical.

34. The apparatus of claim 22 wherein said portion of said second region is a lens.

35. The apparatus of claim 34 wherein said lens is a single element lens.

36. The apparatus of claim 35 wherein said lens is formed integral with said spectacle carrier lens.

37. The apparatus of claim 35 wherein said lens is a stick-on lens applied to the surface of said spectacle carrier lens.

38. The apparatus of claim 23 wherein said lens is inserted into said spectacle carrier lens.

39. The apparatus of claim 23 wherein said carrier lens includes an optical prescription to correct the vision of the corresponding eye.

40. The apparatus of claim 39 wherein the optical prescription to correct the vision of the corresponding eye is placed on a back surface of said carrier lens.

41. The apparatus of claim 39 wherein the optical prescription to correct the vision of the corresponding eye is placed on a front surface of said carrier lens.

42. The apparatus of claim 22 wherein the first optical element is an intraocular lens for implantation within the eye.

43. The apparatus of claim 22 wherein the first optical element is a contact lens for placement on the surface of the eye.

44. The apparatus of claim 22 wherein the first optical element is a pair of optical elements, one for each eye, and wherein the second optical element is a pair of second optical elements one for each eye, wherein binocular vision is provided for in both the unmagnified wide-angle view and the magnified retinal image view.

45. The apparatus of claim 22 wherein the first optical element is a pair of optical elements, one for each eye, and wherein the second optical element is a single second optical element, wherein binocular vision is provided for in the unmagnified wide-angle view and monocular vision is provided in the magnified retinal image view.

46. A kit of parts for improving low vision in the eye, comprising:
a first optical element including an outer annular region having a first optical power and an inner region having a second optical power, said optical element sized and configured for implantation in the eye;
a second optical element external to the eye sized and configured to be placed in optical communication with said first optical element, said second optical element including first and second regions, said first region having an optical power selected in conjunction with said outer region of said first optical element to provide for unmagnified and unrestricted peripheral vision, said second region having an optical power selected in conjunction with said inner region of said first optical element to provide a magnified retinal image; and
educational materials containing instructions on the application and use of said kit.
